(19) United States
(12) Patent Application Publication
(10) Pub. No.: US 2021/0140959 A1
(43) Pub. Date: May 13, 2021

(54) METHOD FOR PROCESSING DATA FROM POINT-OF-CARE TEST READER

(71) Applicant: NANOBIOLIFE INC., Jeju-si (KR)

(72) Inventors: Soo Kyung KIM, Jeju-si (KR); In Jung KIM, Seoul (KR)

(21) Appl. No.: 17/078,444

(22) Filed: Oct. 23, 2020

(30) Foreign Application Priority Data
Nov. 13, 2019 (KR) 10-2019-0145447

(51) Int. Cl. G01N 33/558; G06K 9/62; G06T 7/00

(52) U.S. Cl. CPC G01N 33/558; G06T 2207/30004; G06T 7/0012; G06K 9/6215

(57) ABSTRACT

Provided is a method for processing data from a point-of-care test reader, which reduces an error of a measured value of the point-of-care test reader for a diagnostic kit to acquire quantitative data having high reliability, and the method includes correcting location errors of a test signal line and a control signal line on a membrane based on scan line signal data obtained by scanning a specific region of the membrane within a diagnostic kit with a laser beam, setting a region of interest for the test signal line and the control signal line of the scan line signal data, and arranging and analyzing each of the scan line signal data in the region of interest.

### TECHNICAL FIELD

The present disclosure relates to a method for processing data from a point-of-care test reader for a diagnostic kit, and more specifically, to a method for processing data from a point-of-care test reader, which reduces a measured value error of the point-of-care test reader for a diagnostic kit to acquire quantitative data having high reliability.

### BACKGROUND ART

A point-of-care test (POCT) refers to a clinical pathology testing performed at a place close to a location in which a patient is cared, and is also referred to as a near patient testing or a bedside testing, a doctor's office testing, a divided testing, or the like. In addition, the point-of-care test is called a bedside testing and is also defined as a testing which may be quickly conducted without pre-processing a specimen such as centrifugation near a person to be tested (patient) and thus used for diagnosis and treatment.

An example of an apparatus for the bedside testing is disclosed in Korean Patent Nos. 10-1363030 (Feb. 7, 2014) and 10-1779087 (Sep. 11, 2017).

Meanwhile, the point-of-care test has advantages which may conduct the testing with a small amount of specimens to obtain quick results, and quickly and correspondingly conduct the treatment through the quick results. In addition, the point-of-care test has an advantage in that the patient may directly conduct the test at home or the like by oneself.

However, there are problems in that the point-of-care test has a high cost, reduced precision and accuracy of a point-of-care test machine, and particularly, the reliability of the measured value error is reduced due to an error of the diagnostic kit itself used for the point-of-care test and an error caused when the diagnostic kit is set to or inserted into the point-of-care diagnostic machine.

### DISCLOSURE

### Technical Problem

The present disclosure is intended to solve the aforementioned problems of the related art, and an object of the present disclosure is to provide a method for processing data from a point-of-care test reader which reduces an error of a measured value of the point-of-care test reader for a diagnostic kit to acquire quantitative data having high reliability.

### Technical Solution

A method for processing data from a point-of-care test reader according to one aspect of the present disclosure for achieving the object includes: correcting location errors of a test signal line and a control signal line on a membrane based on scan line signal data obtained by scanning a specific region of the membrane within a diagnostic kit with a laser beam; setting a region of interest for the test signal line and the control signal line of the scan line signal data; and arranging and analyzing each of the scan line signal data in the region of interest.

In an exemplary embodiment, the correcting includes: finding a signal peak point of the scan line signal data in each section of the scan line on the membrane evenly divided into the number of test signal lines and control signal lines; selecting a section of interest of the maximum peak point among the signal peak points; calculating an error distance based on the evenly divided distance of the section of interest; and moving the scan line signal data for each scan line by the error distance with respect to a center line of the section of interest.

In the exemplary embodiment, the setting includes: measuring slopes for signal points of preset resolutions from the scan line signal data having the maximum peak point; obtaining a temporary left boundary and a temporary right boundary in the region of interest based on the slopes; finding a point having the smallest value of the scan line signal data in each of a preset sectional range leftward from the temporary left boundary and a preset sectional range rightward from the temporary right boundary; and selecting the points having the smallest values of the scan line signal data as a left boundary and a right boundary, respectively.

In the exemplary embodiment, the arranging and analyzing includes: converting a graph connecting the values of the scan line signal data into an approximate curve using an average of the consecutive signal values in a predetermined section; determining a region of effect of the scan line signal data having a predetermined width or a predetermined strength based on a peak value in each section sorted by the peak value of the region of interest; and outputting final data for quantification by obtaining an average of data values of the respective regions of effect for each scan line.

### Advantageous Effects

When the aforementioned method for processing the data from the point-of-care test reader is used, it is possible to reduce the error of the measured value of the point-of-care test reader for the diagnostic kit, thereby acquiring the quantitative data having high reliability.

The present disclosure may reliably quantify and acquire the data from the point-of-care test reader in the bedside testing using the diagnostic kit used for immunological diagnosis or the like, and improve the reliability and accuracy of the point-of-care test through the acquired quantitative data.

### DESCRIPTION OF DRAWINGS

FIG. 1 is a flowchart illustrating a method for processing data from a point-of-care test reader according to an exemplary embodiment of the present disclosure.

FIG. 2 is a diagram for explaining scan line signal data of a diagnostic kit which may be employed the method illustrated in FIG. 1.

FIG. 3 is a diagram for explaining a location error of a membrane of the diagnostic kit illustrated in FIG. 2.

FIG. 4 is a diagram for explaining a location error when the diagnostic kit illustrated in FIG. 2 is placed on a worktable of the point-of-care test reader.

FIG. 5 is a diagram for explaining a process of correcting the location error in the method illustrated in FIG. 1.

FIG. 6 is a diagram for explaining a process of setting a region of interest in the method illustrated in FIG. 1.

FIG. 7 is a diagram for explaining a process of arranging and analyzing the signal data in the method illustrated in FIG. 1.

FIG. 8 is a block diagram illustrating a device for processing data of a point-of-care test reader according to another exemplary embodiment of the present disclosure.

FIG. 9 is a diagram for explaining a reliability level of the method for processing data of the point-of-care test reader according to the present disclosure.

FIG. 10 is a diagram for explaining a reliability level per concentration by the method for processing data of the point-of-care test reader according to the present disclosure.

### BEST MODE

Unless defined otherwise in the present specification, all terms including technical terms and scientific terms used herein have the same meaning as commonly understood by those skilled in the art to which the present disclosure pertains. Terms such as ones defined in the dictionary commonly used should be interpreted to have a meaning consistent with the context meaning of the related technology, and should not be interpreted in an ideal or excessively formal sense unless clearly defined in the present specification.

Hereinafter, preferred exemplary embodiments according to the present disclosure will be described in detail with reference to the accompanying drawings.

FIG. 1 is a flowchart illustrating a method for processing data from a point-of-care test reader according to an exemplary embodiment of the present disclosure. FIG. 2 is a diagram for explaining scan line signal data of a diagnostic kit which may be employed in the method illustrated in FIG. 1. FIG. 3 is a diagram for explaining a location error of a membrane of the diagnostic kit illustrated in FIG. 2. FIG. 4 is a diagram for explaining a location error when the diagnostic kit illustrated in FIG. 2 is placed on a worktable of the point-of-care test reader.

Referring to FIG. 1, a method for processing data from a point-of-care test reader according to the present exemplary embodiment first corrects a location error of a test signal line T and a control signal line C on a membrane from scan line signal data obtained by scanning a specific region of the membrane within a diagnostic kit with a laser beam (S10).

As illustrated in FIG. 2, a scan line (SL) may include a path through which the laser beam of a point-of-care test reader passes on a membrane 14 exposed to a window 12 of a diagnostic kit 10. A plurality of scan lines SL may be formed by a scan operation of the point-of-care test reader for the membrane 14 of the diagnostic kit 10, and scan line signal data may be generated for each scan line.

The scan line signal data are used for obtaining a predetermined test result from the test signal line T and the control signal line C. Meanwhile, the reliable scan line signal data is required for the reliable point-of-care test, but as illustrated in FIG. 3, the reliability of the scan line signal data may be reduced by a location error t1 of the membrane 14 of the diagnostic kit 10 itself, and a mounting location error t2 when the diagnostic kit 10 illustrated in FIG. 4 is placed on the worktable of a point-of-care test reader 20. Therefore, the present exemplary embodiment eliminates the aforementioned location errors through the aforementioned process of correcting the location error.

Next, a region of interest for the test signal line T and the control signal line C of the scan line signal data is set (S20).

Subsequently, each of the scan line signal data is arranged and analyzed in the region of interest (S30).

A step of each component described above will be described in more detail as follows.

FIG. 5 is a diagram for explaining a process of correcting a location error in the method illustrated in FIG. 1.

As illustrated in FIG. 5, the correcting of the location error (see the S10 in FIG. 1) finds a signal peak point of the scan line signal data in each section of the scan line on the membrane evenly divided into the number (n) of test signal lines and control signal lines. The signal peak point of the scan line signal data in each section may be indicated as p0, p1, p2.

Here, when the evenly divided length of the membrane is defined as L, the length of each section is L/n. A boundary 32 of each section is indicated by a dotted line, and the signal peak points p0, p1, p2 are indicated by a solid line (see 34). The signal peak points p0, p1, p2 may be related to the control signal line, the test signal line, and another signal line.

Next, the section of interest having the maximum peak point among the signal peak points is selected. In the present step, the section of interest is a section having the maximum peak point p1.

Next, an error distance d is calculated based on the evenly divided distance L/n and the section boundary 32 of both sides of the maximum peak point location in the section of interest. In the present step, the error distance d may correspond to a difference of an interval between a location of a half of the section interval at the maximum peak point and the boundary 32 of each section.

Next, the scan line signal data is moved for each scan line by the error distance d with respect to a center line 34 of the section of interest. In the present step, the scan line signal data is moved time-series or spatially by the error distance d within the section of interest, such that the center line 34 may be sorted on the center of the section of interest, that is, the middle of the boundary 32 of both sides of the section of interest.

FIG. 6 is a diagram for explaining a process of setting a region of interest in the method illustrated in FIG. 1.

As illustrated in FIG. 6, the setting of the region of interest measures slopes of signal points having preset resolutions from the scan line signal data having the maximum peak point.

Next, a temporary left boundary L and a temporary right boundary R in the region of interest are obtained based on the measured slopes.

Next, a point having the smallest values of the scan line signal data is found from each of a preset section range leftward from the temporary left boundary L and a preset section range rightward from the temporary right boundary R.

Next, the points having the smallest value of the scan line signal data in the preset section range are selected as a left boundary L′ and a right boundary R′, respectively.

The present exemplary embodiment may obtain a temporary boundary having the largest slope at the present resolution around the peak point from the consecutive signal values of the scan line signal data, and based on the temporary boundary, set a primary boundary of the data in the region of interest to be used for the post-analysis based on a substantial signal value.

FIG. 7 is a diagram for explaining a process of arranging and analyzing the signal data in the method illustrated in FIG. 1.

As illustrated in FIG. 7, the arranging and analyzing of the scan line signal data first converts a graph connecting the values of the scan line signal data into an approximate curve using an average of the consecutive signal values in a predetermined section. The approximate curve conversion process may be performed in a method for setting 3 to 12 data points of the consecutive data as sectional data and obtaining an average of the sectional data (see FIGS. 7A and 7B). According to the approximate curve conversion process, the curve of the scan line signal data may be smooth, and the signal value may be slightly reduced.

Next, a region of effect (ROE) of the scan line signal data having a predetermined width or a predetermined strength based on the peak value in each section sorted by the peak value of a region of interest (ROI) is determined. A ROI1 may be a region of interest of the control signal line, and a ROI2 may be a region of interest of the test signal line, and vice versa.

The present step removes substantially ineffective data at the boundary of each signal line when a spot region of the laser beam is close to the data signal line or the control signal line upon movement along the scan line and passes over the data signal line or the control signal line, and the present process may limit data to be analyzed to only data having better quality to reduce capacity, thereby providing quantitative data having high reliability.

Next, final data for quantification may be output by obtaining an average of the data values of the respective regions of effect for each scan line. The final data is not limited to obtaining the average of the data of the respective regions of effect, and various methods for obtaining the median value or obtaining an average of values in the median range may be replaced and used.

FIG. 8 is a block diagram illustrating a device for processing data of a point-of-care test reader according to another exemplary embodiment of the present disclosure.

Referring to FIG. 8, the device for processing data of the point-of-care test reader 100 (hereinafter, simply referred to as ‘data processing device’) according to the present exemplary embodiment includes a processor 110, a memory 120, and an interface 150.

The processor 110 includes a microprocessor, and may execute and mount a program or a software module stored in the memory 120, and perform a series of procedures for the method for processing the data from the point-of-care test reader described above with reference to FIGS. 1 to 7 through the mounted software module.

The memory 120 may store a location error correction unit 120a, a region of interest setting unit 120b, and a signal data arrangement and analysis unit 120c. In addition, the memory 120 may store a peak point tracking module 121, a section of interest selection module 122, an error distance calculation module 123, and a signal data movement module 124, and the modules 121 to 124 may be provided in the error correction unit 120a.

In addition, the memory 120 may store a boundary tracking module 125, a minimum signal value tracking module 126, and a boundary selection module 127, and the modules 125 to 127 may be provided in the region of interest setting unit 120b.

In addition, the memory 120 may store an approximate curve conversion module 128, a region of effect determination module 129, and an average calculation module 130, and the modules 128 to 130 may be provided on the signal data arrangement and analysis unit 120c.

The interface 150 may include an inner communication interface, a communication interface, a user interface, or a combination thereof. The interface 150 may be connected to the processor 110 and connected to an input and output devices or a network to transmit and receive signals and data.

The aforementioned data processing device may be implemented by the point-of-care test reader or a computing device or a server device connected to the point-of-care test reader via a network. The network may include a wired network, a wireless network, a near field wireless communication network, an intranet, an Internet, a satellite network, or the like.

FIG. 9 is a diagram for explaining a reliability level of the method for processing the data from the point-of-care test reader according to the present disclosure. In addition, FIG. 10 is a diagram for explaining a reliability level per concentration by the method for processing the data from the point-of-care test reader according to the present disclosure.

FIGS. 9 and 10 illustrate that the method for processing the data from the point-of-care test reader according to the present exemplary embodiment has significantly high reliability compared to a conventional method for processing data from a point-of-care test reader using a camera (Comparative Example).

That is, the present exemplary embodiment uses a ratio of the test signal value to the control signal value (T/C ratio) for the quantification analysis and compares the result thereof with a Comparative Example. In the experiment, before the signal value is measured, antigen is diluted at each concentration and the signal value is measured by each reader. Insertion shows the inversely calculated dose.

It was shown that a coefficient of determination R² in the present exemplary embodiment was 0.998, and was much higher than 0.967 which is a coefficient of determination in the Comparative Example.

In addition, the standard deviation of the signal value ratios (T/C ratio) to three antigen concentration points (high, medium, low) in the present exemplary embodiment was calculated lower than that of the Comparative Example in all concentration ranges.

As described above, it was confirmed that the point-of-care test reader according to the present exemplary embodiment senses signals at both low and high concentrations more accurately than the camera type reader (Comparative Example) and converts the signals into values close to the actual concentration. As a result, it may be seen that the data processing method according to the present exemplary embodiment may have a wider dynamic range with a uniform signal value interval distribution over all concentration ranges.

Although the present disclosure has been described above with reference to the preferred exemplary embodiment of the present disclosure, those skilled in the art will understand that the present disclosure may be modified and changed variously without departing from the spirit and scope of the present disclosure described in the appended claims.

1. A method for processing data from a point-of-care test reader, the method comprising:
correcting location errors of a test signal line and a control signal line on a membrane from scan line signal data obtained by scanning a specific region of the membrane within a diagnostic kit with a laser beam;
setting a region of interest for the test signal line and the control signal line of the scan line signal data; and
arranging and analyzing each of the scan line signal data in the region of interest.

2. The method of claim 1,
wherein the correcting comprises:
finding a signal peak point of the scan line signal data in each section of the scan line on the membrane evenly divided into the number of test signal lines and control signal lines;
selecting a section of interest of the maximum peak point among the signal peak points;
calculating an error distance based on the evenly divided distance of the section of interest; and
moving the scan line signal data for each scan line by the error distance with respect to a center line of the section of interest.

3. The method of claim 2,
wherein the setting comprises:
measuring slopes for signal points of preset resolutions from the scan line signal data having the maximum peak point;
obtaining a temporary left boundary and a temporary right boundary in the region of interest based on the slopes;
finding a point having the smallest value of the scan line signal data in each of a preset sectional range leftward from the temporary left boundary and a preset sectional range rightward from the temporary right boundary; and
selecting the points having the smallest values of the scan line signal data as a left boundary and a right boundary, respectively.

4. The method of claim 3,
wherein the arranging and analyzing comprises:
converting a graph connecting the values of the scan line signal data into an approximate curve using an average of the consecutive signal values in a predetermined section;
determining a region of effect of the scan line signal data having a predetermined width or a predetermined strength based on a peak value in each section sorted by the peak value of the region of interest; and
outputting final data for quantification by obtaining an average of data values of the respective regions of effect for each scan line.
